(19) United States
(12) Patent Application Publication
(10) Pub. No.: US 2022/0356631 A1
(43) Pub. Date: Nov. 10, 2022

(54) SANITATION SYSTEM FOR LAUNDRY TREATING APPLIANCES

(71) Applicant: WHIRLPOOL CORPORATION, Benton Harbor, MI (US)

(72) Inventor: Sayer J. Murphy, St. Joseph, MI (US)

(21) Appl. No.: 17/731,461

(22) Filed: Apr. 28, 2022

Related U.S. Application Data
(60) Provisional application No. 63/185,083, filed on May 6, 2021.

(51) Int. Cl. D06F 39/06; D06F 37/20; D06F 39/14; D06F 39/02; D06F 35/00; D06F 23/04; A61L 2/10; A61L 9/20; A61L 2/20

(52) U.S. Cl. CPC D06F 39/06; D06F 37/20; D06F 39/14; D06F 39/02; D06F 35/001; D06F 23/04; A61L 2/10; A61L 9/20; A61L 2/20; A61L 2202/11; A61L 2209/12; A61L 2202/17

(57) ABSTRACT

A sanitizing system for use in a laundry treating appliance and at least partially included in an agitator of the laundry treating appliance, the agitator may include a housing configured to be removably attached to a rotating drum or an impeller of laundry treating appliance, and an ultraviolet light disposed in the housing, wherein the ultraviolet light is configured to transmit ultraviolet light to articles of clothing disposed in a drum of the washing machine.

### CROSS-REFERENCE TO RELATED APPLICATIONS

This application claims the benefit of U.S. provisional application Ser. No. 63/185,083 filed May 6, 2021, the disclosure of which is hereby incorporated in its entirety by reference herein.

### TECHNICAL FIELD

Aspects of the disclosure generally relate to sanitation systems for laundry treating appliances.

### BACKGROUND

Laundry treating appliances, such as clothes washers, clothes dryers, washing machines, refreshers, and non-aqueous systems, can have a configuration based on a container, such as a laundry basket or drum that defines a drum opening, which may or may not rotate, and that at least partially defines a treating chamber in which laundry items are placed for treating. The laundry treating appliance can have a controller that implements a number of user-selectable, pre-programmed cycles of operation having one or more operating parameters. Hot water, cold water, or a mixture thereof, along with various treating chemistries, or detergents, can be supplied to the treating chamber in accordance with the cycle of operation.

Laundry treating appliances typically operate to treat laundry items by placing the laundry items in contact with treating fluid such as a detergent/water mixture, sometimes referred to as wash liquor, and providing relative motion between the laundry items and the fluid. The clothes mover can include multiple components, such as a base, which can be provided as an impeller plate, and a barrel, which can be provided as an agitator post, and which can couple to the base.

Filters are used in laundry treating appliances to capture pet hair, lint, and other particulate from laundry loads. The captured particulate is retained inside the filter, allowing the consumer to clean the filter after a wash cycle is completed. For customers who do not use clothes drying appliances, it is especially important to trap lint during the wash cycle. It is also important to keep these filters, and the basket or drum clean.

### SUMMARY

A sanitizing system for use in a laundry treating appliance and at least partially included in an agitator of the laundry treating appliance, the agitator may include a housing configured to be removably attached to a rotating drum or an impeller of laundry treating appliance, and an ultraviolet light disposed in the housing, wherein the ultraviolet light is configured to transmit ultraviolet light to articles of clothing disposed in a drum of the washing machine.

In one example, the sanitizing system includes a battery disposed within the housing and configured to power the ultraviolet light.

In one embodiment, the sanitizing system includes a charger configured to receive the housing and charge the battery when the housing is removed from the rotating drum or impeller.

In one example, the charger includes a receptacle configured to engage and receive the housing.

In another example, the receptacle includes a connector configured to engage a portable energy source within the housing.

In a further embodiment, the charger is powered by an AC power source.

A sanitizing system for use in a laundry treating appliance, the sanitizing system may include a treating chemistry dispenser configured to maintain a sanitizing solution and selectively vaporize and distribute the vaporized solution to a treating chamber of the appliance, a fan arranged at a port connecting to the treating chamber to expel vapors from the treating chamber; and a gasket assembly arranged on a lid of the appliance to latch the lid in a sealed and closed position during vaporization.

In one embodiment, the sanitizing system includes a controller programmed to activate the actuation mechanism in response to the laundry treating appliance initiating a tumble cycle.

In one example, the controller is further programmed to activate the actuation mechanism for a predetermined amount of time to sanitize items within the laundry treating appliance.

In a further embodiment, the retention mechanism is a strap configured to selectively receive the container.

A sanitizing system for use in a laundry treating appliance, the system may include a retention mechanism arranged on an inside surface of a door of the laundry treating appliance, an aerosol container containing liquid disinfectant, the container including a spray nozzle, an actuation mechanism configured to selectively actuate the spray nozzle to deliver disinfectant to a treating chamber of the laundry treating apparatus.

In another example, the sanitizing system includes a latch configured to clamp the lid to the cabinet and create a closed systems with a negative pressure to prevent leakage of the vapors from the basket.

In another embodiment, the gasket assembly includes a latch action toggle.

In a further example, the gasket assembly includes a pinch latch.

In another example, the treating chemistry dispenser includes an ozone generator.

In another embodiment, the treating chemistry dispenser includes a vaporized peroxide generator.

### BRIEF DESCRIPTION OF THE DRAWINGS

The embodiments of the present disclosure are pointed out with particularity in the appended claims. However, other features of the various embodiments will become more apparent and will be best understood by referring to the following detailed description in conjunction with the accompany drawings in which:

FIG. 1 illustrates a perspective view of an example laundry treating appliance having a sanitation system;

FIG. 2 illustrates another example laundry treating appliance having another example sanitation system; and

FIG. 3 illustrates a perspective cross-sectional view of another laundry treating appliance having another example sanitization system.

### DETAILED DESCRIPTION

As required, detailed embodiments of the present invention are disclosed herein; however, it is to be understood that the disclosed embodiments are merely exemplary of the invention that may be embodied in various and alternative forms. The figures are not necessarily to scale; some features may be exaggerated or minimized to show details of particular components. Therefore, specific structural and functional details disclosed herein are not to be interpreted as limiting, but merely as a representative basis for teaching one skilled in the art to variously employ the present invention.

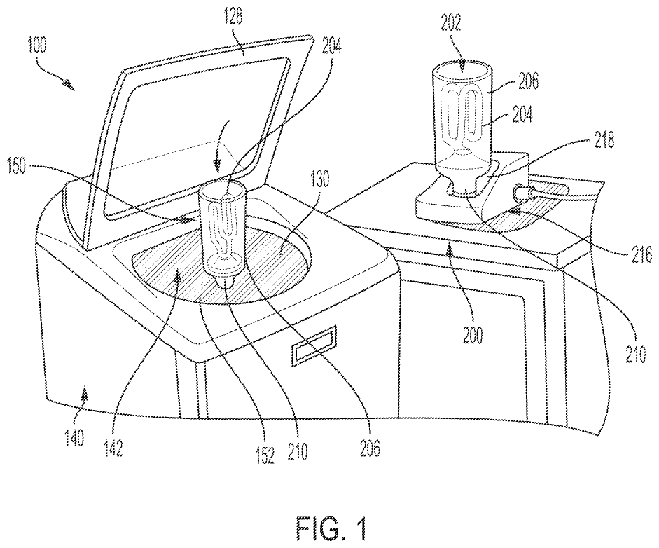

FIG. 1 illustrates an example laundry treating appliance 100 including a removable agitator 150. The removable agitator 150 may be a filtering agitator. The laundry treating appliance 100 can be any laundry treating appliance 100 that performs a cycle of operation to clean or otherwise treat laundry items placed therein, non-limiting examples of which include a horizontal or vertical axis clothes washer; a clothes dryer; a combination washing machine and dryer; a dispensing dryer; a tumbling or stationary refreshing/revitalizing machine; an extractor; a non-aqueous washing apparatus; and a revitalizing machine. While the laundry treating appliance 100 is illustrated herein as a vertical axis, top-load laundry treating appliance 100, the aspects of the present disclosure can have applicability in laundry treating appliances with other configurations. The laundry treating appliance 100 shares many features of a conventional automated clothes washer and/or dryer, which will not be described in detail herein except as necessary for a complete understanding of the exemplary aspects in accordance with the present disclosure.

Laundry treating appliances are typically categorized as either a vertical axis laundry treating appliance or a horizontal axis laundry treating appliance. As used herein, the term “horizontal axis” laundry treating appliance refers to a laundry treating appliance having a rotatable drum that rotates about a generally horizontal axis relative to a surface that supports the laundry treating appliance. The drum can rotate about the axis inclined relative to the horizontal axis, with fifteen degrees of inclination being one example of the inclination. Similar to the horizontal axis laundry treating appliance, the term “vertical axis” laundry treating appliance refers to a laundry treating appliance having a rotatable drum that rotates about a generally vertical axis relative to a surface that supports the laundry treating appliance. However, the rotational axis need not be perfectly vertical to the surface. The drum can rotate about an axis inclined relative to the vertical axis, with fifteen degrees of inclination being one example of the inclination.

In another aspect, the terms vertical axis and horizontal axis are often used as shorthand terms for the manner in which the appliance imparts mechanical energy to the laundry, even when the relevant rotational axis is not absolutely vertical or horizontal. As used herein, the “vertical axis” laundry treating appliance refers to a laundry treating appliance having a rotatable drum, perforate or imperforate, that holds fabric items and, optionally, a clothes mover, such as an agitator, impeller, nutator, and the like within the drum. The clothes mover can move within the drum to impart mechanical energy directly to the clothes or indirectly through wash liquid in the drum. The clothes mover can typically be moved in a reciprocating rotational movement. In some vertical axis laundry treating appliances, the drum rotates about a vertical axis generally perpendicular to a surface that supports the laundry treating appliance. However, the rotational axis need not be vertical. The drum can rotate about an axis inclined relative to the vertical axis.

As used herein, the “horizontal axis” laundry treating appliance refers to a laundry treating appliance having a rotatable drum, perforated or imperforate, that holds laundry items and washes and/or dries the laundry items. In some horizontal axis laundry treating appliances, the drum rotates about a horizontal axis generally parallel to a surface that supports the laundry treating appliance. However, the rotational axis need not be horizontal. The drum can rotate about an axis inclined or declined relative to the horizontal axis. In horizontal axis laundry treating appliances, the clothes are lifted by the rotating drum and then fall in response to gravity to form a tumbling action. Mechanical energy is imparted to the clothes by the tumbling action formed by the repeated lifting and dropping of the clothes. Vertical axis and horizontal axis machines are best differentiated by the manner in which they impart mechanical energy to the fabric articles.

Regardless of the axis of rotation, a laundry treating appliance can be top-loading or front-loading. In a top-loading laundry treating appliance, laundry items are placed into the drum through an access opening in the top of a cabinet, while in a front-loading laundry treating appliance laundry items are placed into the drum through an access opening in the front of a cabinet. If a laundry treating appliance is a top-loading horizontal axis laundry treating appliance or a front-loading vertical axis laundry treating appliance, an additional access opening is located on the drum.

In more detail, the laundry treating appliance 100 can include a structural support assembly comprising a cabinet 140, which defines a housing and an interior, within which a laundry holding assembly resides. The cabinet 140 can be a housing having a chassis and/or a frame, to which decorative panels can or cannot be mounted, defining an interior, enclosing components typically found in a conventional laundry treating appliance, such as an automated clothes washer or dryer, which can include motors, pumps, fluid lines, controls, sensors, transducers, and the like. Such components will not be described further herein except as necessary for a complete understanding of the present disclosure.

The laundry holding assembly of the illustrated exemplary laundry treating appliance 100 can include a rotatable basket 130 having an open top that can be disposed within the interior of the cabinet 140 and can at least partially define a rotatable treating chamber 142 for receiving laundry items for treatment and an access opening 152. The access opening 152 can provide access to the treating chamber. The treating chamber 142 is configured to receive a laundry load comprising laundry items for treatment, including, but not limited to, a hat, a scarf, a glove, a sweater, a blouse, a shirt, a pair of shorts, a dress, a sock, and a pair of pants, a shoe, an undergarment, a jacket, bedding, blankets, rugs, pillows, etc.

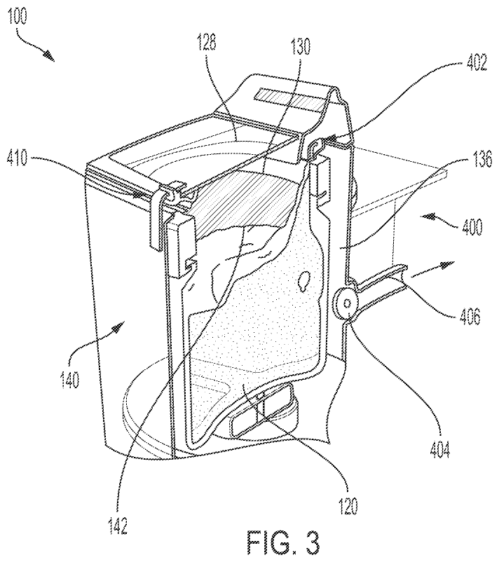

As best illustrated in FIG. 3, a tub 134 can also be positioned within the cabinet 140 and can define an interior within which the basket 130 can be positioned. The tub 134 can also at least partially define at least a portion of the treating chamber 142. The tub can be at least partially aligned with the access opening 152 (not labeled in FIG. 3) and the open top. In one example, the tub 134, the basket 130, and the access opening 152, can have central axes that are co-axial with one another, or with at least one of the other axes, such that a common central axis is formed.

The basket 130 can have a generally peripheral side wall, which is illustrated as a cylindrical side wall, closed at the basket end by a basket base 120 to further at least partially define the treating chamber 142. The basket 130 can be rotatably mounted within the tub 134 for rotation about a vertical basket axis of rotation and can include a plurality of perforations (not shown), such that liquid can flow between the tub 134 and the rotatable basket 130 through the perforations (not shown). While the illustrated laundry treating appliance 100 includes both the tub 134 and the basket 130, with the basket 130 at least partially defining the treating chamber 142, it is also within the scope of the present disclosure for the laundry holding assembly to include only one receptacle, such as the tub 134, without the basket 130, with the receptacle defining the laundry treating chamber 142 for receiving the load to be treated.

A selectively openable closure or cover, illustrated herein as comprising a lid 128, can be movably mounted to or coupled to the cabinet 140 for selective movement between an opened position and a closed position, as shown, to selectively open and close the access opening 152, respectively, and to selectively provide access into the laundry treating chamber 142 through the access opening 152 of the basket 130. In one example, the lid 128 can be rotatable between the closed position and the opened position relative to the cabinet 140. By way of non-limiting example, the lid 128 can be hingedly coupled to the cabinet 140 for movement between the opened position and the closed position. In the closed position, the lid 128 can seal against at least one of the access opening 152, and can at least partially confront the treating chamber 142 when the lid 128 closes the access opening 152. In the opened position, the lid 128 can be spaced apart from the access opening 152, and can allow access to the access opening 152.

Referring back to FIG. 1, a clothes mover (not illustrated in the figures) can be rotatably mounted within the basket 130 to impart mechanical agitation and energy to a load of laundry items placed in the basket 130 or the treating chamber 142 according to a cycle of operation. The clothes mover can be oscillated or rotated about its vertical axis of rotation during a cycle of operation in order to produce load motion effective to wash the load contained within the treating chamber 142. The clothes mover can comprise a base or a first clothes mover, such as an impeller, and a barrel, forming the removable agitator 150. The agitator 150 as illustrated herein can comprise a vertically oriented agitator post that can be removably coupled with the impeller (not shown), the agitator 150 projecting vertically from the impeller within the treating chamber 142 and toward the open top of the basket 130.

The agitator 150 can include any configuration of vanes, blades, or other structural features for imparting mechanical energy to laundry items during a cycle of operation. Generally, the vertical extent of the agitator 150, combined with vane, blade, or other structural features, can impart the mechanical action to laundry items, which provides improved cleaning performance and can be suitable for particularly soiled loads. Other exemplary types of clothes movers include, but are not limited to, an agitator alone, a wobble plate, and a hybrid impeller/agitator.

The laundry treating appliance 100 may include, or at least partially include, a sanitation system 200 configured to provide sanitizing techniques to the treating chamber 142. The sanitation system 200 may include the agitator 150, which includes a sanitation component 202 housed in a housing 206. The housing 206 may be a canister configured to fit within the agitator 150 or be integrated within the agitator 150.

The sanitation component 202 may include a component configured to sanitize contents of the treating chamber 142. In one example, the sanitation component 202 may be an ultra-violet (UV) light 204 device, such as a UV light bulb. The UV light 204 may be configured to act as disinfectant for air, water, surfaces, items, etc., to reduce or kill bacteria, viruses, or pathogens. The UV light 204 may produce UV rays that destroy certain molecular bonds that maintain the DNA of viruses and bacteria. The UV light may produce UVA, UVB, or UVC rays, with UVC being the more effective of the three at destroying pathogens.

When UV rays come into contact with the treating chamber 142 of the laundry treating appliance 100, the UV rays may disinfect each surface exposed to the rays. That is, the contents within the treating chamber 142, as well as the treating chamber 142, agitator 150, basket, lid 128, etc., may each be disinfected of any pathogens. By arranging the UV light 204 within the agitator 150, the UV light 204 may arranged in the center of the basket 130 and treating chamber 142 and thus provide UV rays to a large constituent of the interior of the appliance 100.

The UV light 204 may be powered by a portable energy source 210 such as a battery. The energy source 210 may be a rechargeable battery. In the example of FIG. 1, the sanitation system 200 includes a charger 216 separate from the laundry treating appliance 100. The charger 216 may include a receptacle 218 configured to engage and receive the agitator 150 and/or the housing 206 in the event the housing 206 is removable from the agitator 150. The receptacle 218 may include a connector (not shown) configured to engage either directly or indirectly with the portable energy source 210 when the agitator 150 is seated in the charger 216. The connectors may transmit power from the charger 216 to the battery, thus charging the battery. The charger 216 may be powered by an AC power source such as wall-outlet. The energy source 210 may be arranged at a first end of the agitator 150 so as to easily abut the connectors when received within the receptacle 218. The sanitation component 202 may connect within the agitator 150 in a similar manner as to its seating within the charger.

The sanitation system 200 may be an aftermarket system configured to be sold separate from the laundry treating appliance 100. Additionally or alternatively, the sanitation system 200 may be integral to the laundry treating appliance 100. The rechargeability of the removable agitator 150 allows for multiple and long-term use of the sanitation system. By including the sanitation component 202 within the removable agitator 150, the sanitation system 200 is easily accessible, easily implemented as an accessory and aftermarket product, easily reusable, as well as providing a wide range of sanitation to the treating chamber 142. Notably, while the agitator 150 is described herein as being a removable agitator 150, the agitator may also be fixed within the basket 130 and the housing 206 may be removable from the agitator 150.

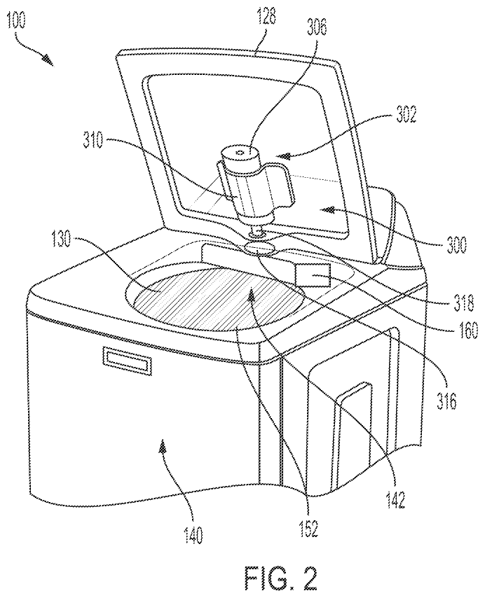

FIG. 2 illustrates a partial perspective view of the laundry treating appliance 100 having another example sanitation system 300. In this example, the sanitation system 300 may include an aerosol sanitizer 302 arranged on an inside of the lid 128 or door of the appliance 100. The lid 128 is illustrated in an open position FIG. 2 for ease of explanation. While a top loading appliance 100 is illustrated, a front loading appliance with a hinged door may also be appreciated with respect to this example.

The sanitizer 302 may include an aerosol can 306 or container configured to hold sanitizing solutions configured to kill viruses and bacteria. For example, the solutions may include disinfectant solutions such as bleach, alcohol, baking soda, ammonia, vinegar, soap, detergent, rinse, etc. The aerosol can 306 may be held in place by a retention mechanism 310 or strap, allowing the can to be selectively inserted therein. The strap may also allow easy access for replacing or replenishing the can.

The sanitation system 300 includes an actuation mechanism 316 controlled by a controller 160, as shown in FIG. 1, that actuates a spray nozzle 318 of the aerosol can 306. Upon actuation, the sanitizing solution may be sprayed around and within the treating chamber 142 to disinfect the contents therein. The controller 160 may be configured to, responsive to the appliance 100 activating a tumble cycle, actuate the actuation mechanism 316 for a predetermined amount of time to sanitize clothes, or other items within the treatment chamber. In one example, the predetermined amount of time may be approximately two minutes.

The controller 160 may be used to trigger activation of the sanitation system 300 based on a certain operating status or mode of the appliance 100. The controller 160 may be coupled with various working components of the laundry treating appliance 100 to control the operation of the working components and to implement one or more treating cycles of operation. The control assembly can include the controller 160 located within the cabinet 140 and a user interface that can be operably coupled with the controller 160. The user interface can provide an input and output function for the controller.

The user interface can include one or more knobs, dials, switches, displays, touch screens and the like for communicating with the user, such as to receive input and provide output. For example, the displays can include any suitable communication technology including that of a liquid crystal display (LCD), a light-emitting diode (LED) array, or any suitable display that can convey a message to the user. The user can enter different types of information including, without limitation, cycle selection and cycle parameters, such as cycle options. Other communications paths and methods can also be included in the laundry treating appliance 100 and can allow the controller 160 to communicate with the user in a variety of ways. For example, the controller can be configured to send a text message to the user, send an electronic mail to the user, or provide audio information to the user either through the laundry treating appliance 100 or utilizing another device such as a mobile phone. The user may activate the sanitations systems described herein via the user interface.

The controller 160 can include the machine controller and any additional controllers provided for controlling any of the components of the laundry treating appliance 100. For example, the controller 160 can include the machine controller and a motor controller. Many known types of controllers can be used for the controller 160. It is contemplated that the controller 160 is a microprocessor-based controller that implements control software and sends/receives one or more electrical signals to/from each of the various working components to implement the control software. As an example, proportional control (P), proportional integral control (PI), and proportional derivative control (PD), or a combination thereof, a proportional integral derivative control (PID), can be used to control the various components of the laundry treating appliance 100.

FIG. 3 illustrates a perspective cut-away view of the laundry treating appliance 100 having another example sanitation system 400. The sanitation system 400 may include treating chemistry dispenser 402 can be provided for dispensing treating chemistry to the basket 130 for use in sanitizing the laundry items. The treating chemistry dispenser 402 can be a single use dispenser, a bulk dispenser, or a combination of or an integrated single use and bulk dispenser, in non-limiting examples, and is fluidly coupled to the treating chamber 142. While the treating chemistry dispenser 402 is illustrated herein as being provided at a top panel or a shroud, it will be understood that other locations for the treating chemistry dispenser 402 can be contemplated, such as at a different location within the cabinet 140.

The treating chemistry dispenser 402 may be configured to house sanitizing liquid such as peroxide. Similar to the above examples, the sanitizing liquid is configured to be detrimental to the survival of viruses and bacteria and provides sanitation of items it comes into contact with. The treating chemistry dispenser 402 may include an ozone generator or vaporizer configured to vaporize the peroxide. During a wash cycle, the treating chemistry dispenser 402 may disperse vaporized hydrogen peroxide within the treating chamber 142 to sanitize the contents within the basket 130.

The sanitation system 400 may include at least one fan 404 arranged on a port 406. The fan 404 is configured to create a negative pressure in the cabinet to prevent any vapors from leaking into the room. The fan 404 may expel the vapors from the basket 130. A gasket assembly 410 may be arranged on the appliance lid 128 to seal the lid 128 during vaporization of the sanitizing agent. The gasket assembly 410 may include a gasket and a pinch latch, or a latch action toggle clamp configured to clamp the lid 128 to the cabinet 140. By latching the lid 128, a closed system is created, allowing the fan to create the negative pressure and thus preventing any leakage of the vapors from the basket 130.

The gasket assembly 410 may be manually latched by the user, or may be controlled by the controller 160. In the example of a manual latch, a sensor may be arranged at or near the lid 128 to detect whether the lid 128 is fully latched. Vaporization may be unavailable until the sensor indicates that the lid 128 is fully latched. The controller 160 may also control the fan 404 and the treating chemistry dispenser 402 to dispense and vaporize the peroxide. Under certain conditions the peroxide may be vaporized, such as in response to the lid 128 being latched and the appliance 100 operating in a wash cycle.

The treating chemistry dispenser 402 may also be used for detergent to or with water from a water supply during the wash cycle. The treating chemistry dispenser 402 can include multiple chambers or reservoirs fluidly coupled to the treating chamber 142 for receiving doses of different treating chemistries (e.g., detergent and peroxide separately).

Additionally, the liquid supply assembly and treating chemistry dispenser 402 can differ from the configuration shown, such as by inclusion of other valves, conduits, wash aid dispensers, heaters, sensors, such as water level sensors and temperature sensors, and the like, to control the flow of treating liquid through the laundry treating appliance 100 and for the introduction of more than one type of detergent and sanitizing agent.

The descriptions of the various embodiments have been presented for purposes of illustration, but are not intended to be exhaustive or limited to the embodiments disclosed. Many modifications and variations will be apparent to those of ordinary skill in the art without departing from the scope and spirit of the described embodiments.

Aspects of the present embodiments may be embodied as a system, method or computer program product. Accordingly, aspects of the present disclosure may take the form of an entirely hardware embodiment, an entirely software embodiment (including firmware, resident software, micro-code, etc.) or an embodiment combining software and hardware aspects that may all generally be referred to herein as a “module” or “system.” Furthermore, aspects of the present disclosure may take the form of a computer program product embodied in one or more computer readable medium(s) having computer readable program code embodied thereon.

Any combination of one or more computer readable medium(s) may be utilized. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium include the following: an electrical connection having one or more wires, a portable computer diskette, a hard disk, a random access memory (RAM), a read-only memory (ROM), an erasable programmable read-only memory (erasable programmable read-only memory (EPROM) or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

Aspects of the present disclosure are described above with reference to flowchart illustrations and/or block diagrams of methods, apparatus (systems) and computer program products according to embodiments of the disclosure. It will be understood that each block of the flowchart illustrations and/or block diagrams, and combinations of blocks in the flowchart illustrations and/or block diagrams, can be implemented by computer program instructions. These computer program instructions may be provided to a processor of a general-purpose computer, special purpose computer, or other programmable data processing apparatus to produce a machine, such that the instructions, which execute via the processor of the computer or other programmable data processing apparatus, enable the implementation of the functions/acts specified in the flowchart and/or block diagram block or blocks. Such processors may be, without limitation, general purpose processors, special-purpose processors, application-specific processors, or field-programmable.

While exemplary embodiments are described above, it is not intended that these embodiments describe all possible forms of the invention. Rather, the words used in the specification are words of description rather than limitation, and it is understood that various changes may be made without departing from the spirit and scope of the invention. Additionally, the features of various implementing embodiments may be combined to form further embodiments of the invention.

What is claimed is:

1. A sanitizing system for use in a laundry treating appliance and at least partially included in an agitator of the laundry treating appliance, the agitator comprising:
a housing configured to be removably attached to a rotating drum or an impeller of a laundry treating appliance; and
an ultraviolet light disposed in the housing, wherein the ultraviolet light is configured to emit ultraviolet light onto articles of clothing disposed in a drum of the laundry treating appliance.

2. The sanitizing system of claim 1, further comprising a battery disposed within the housing and configured to power the ultraviolet light.

3. The sanitizing system of claim 2, further comprising a charger configured to receive the housing and charge the battery when the housing is removed from the rotating drum or impeller.

4. The sanitizing system of claim 3, wherein the charger includes a receptacle configured to engage and receive the housing.

5. The sanitizing system of claim 4, wherein the receptacle includes a connector configured to engage a portable energy source within the housing.

6. The sanitizing system of claim 4, wherein the charger is powered by an AC power source.

7. A sanitizing system for use in a laundry treating appliance, the system comprising:
a retention mechanism arranged on an inside surface of a door of the laundry treating appliance;
an aerosol container containing liquid disinfectant, the container including a spray nozzle; and
an actuation mechanism configured to selectively actuate the spray nozzle to deliver disinfectant to a treating chamber of the laundry treating appliance.

8. The sanitizing system of claim 7, further comprising a controller programmed to activate the actuation mechanism in response to the laundry treating appliance initiating a tumble cycle.

9. The sanitizing system of claim 8, wherein the controller is further programmed to activate the actuation mechanism for a predetermined amount of time to sanitize items within the laundry treating appliance.

10. The sanitizing system of claim 7, wherein the retention mechanism is a strap configured to selectively receive the container.

11. A sanitizing system for use in a laundry treating appliance, the sanitizing system comprising:
a treating chemistry dispenser configured to maintain a sanitizing solution and selectively vaporize and distribute the vaporized solution to a treating chamber of the laundry treating appliance;
a fan arranged at a port connecting to the treating chamber to expel vapors from the treating chamber; and
a gasket assembly arranged on a lid of the appliance to latch the lid in a sealed and closed position during vaporization.

12. The sanitizing system of claim 11, further comprising a latch configured to clamp the lid to a cabinet of the appliance and create a closed system with a negative pressure to prevent leakage of the vapors from a basket of the appliance.

13. The sanitizing system of claim 11, wherein the gasket assembly includes a latch action toggle.

14. The sanitizing system of claim 11, wherein the gasket assembly includes a pinch latch.

15. The sanitizing system of claim 11, wherein the treating chemistry dispenser includes an ozone generator.

16. The sanitizing system of claim 11, wherein the treating chemistry dispenser includes a vaporized peroxide generator.
